(19) United States
(12) Patent Application Publication
(10) Pub. No.: US 2026/0256521 A1
(43) Pub. Date: Sep. 3, 2026

(54) INTRAOPERATIVE SURGICAL RESECTION PLANNING

(71) Applicant: STA Innovations, LLC., Sammamish, WA (US)

(72) Inventors: Timothy B. ALTON, Kent, WA (US); Philip G. HUJDIC, Kirkland, WA (US)

(73) Assignee: STA Innovations, LLC, Sammamish, WA (US)

(21) Appl. No.: 19/659,438

(22) Filed: Apr. 27, 2026

Related U.S. Application Data
(63) Continuation of application No. PCT/US2024/053340, filed on Oct. 29, 2024.
(60) Provisional application No. 63/595,133, filed on Nov. 1, 2023.
(60) Provisional application No. 63/635,826, filed on Apr. 18, 2024.

(51) Int. Cl. A61B 34/10; A61B 34/00

(52) U.S. Cl. CPC A61B 34/10; A61B 34/25; A61B 2034/104; A61B 2034/107; A61B 2034/252

(57) ABSTRACT

Intraoperative surgical resection planning includes setting a cutting angle for a planned distal femur resection to provide a desired extension gap relation, automatically setting distal femur resection amounts for the planned distal femur resection such that a medial or lateral extension gam is within a threshold of a target, planning a femoral component rotation to provide a desired flexion gap relation, selecting a femoral component size based on flexing/extending a current planned femoral component, determining whether the desired extension gap relation is maintained, and performing processing based on whether the desired extension gap relation is maintained.

### BACKGROUND

The use of robotic-assisted surgical joint replacement technology can be helpful in some regards but has drawbacks. The process is often complicated, has a steep learning curve, and requires extensive training and multiple steps of surgeon deliberation, contemplation, and decision-making. Additionally, it is non-universal in that it can be difficult to identify pre-operative targets. Current techniques are not standardized, and targets change from case-to-case. It is also inconsistent as there is vast variability in both the approach and the clinical results, and inefficient due to excessive time spent manually manipulating intraoperative data while in surgery. It often requires manual manipulation from a skilled professional, such as an industry representative (e.g., one hired by a vendor company at an added cost) or dedicated operating room staff.

### SUMMARY

Shortcomings of the prior art are overcome and additional advantages are provided. In an embodiment, a computer-implemented method is provided for intraoperative surgical resection planning of a surgery to a knee joint of a patient, the knee joint including a patient femur and patient tibia. The method includes setting a cutting angle for a planned distal femur resection of the patient femur that provides a desired extension gap relation between a medial extension gap (Em) on a medial side of the patient femur between the patient femur and patient tibia and a lateral extension gap (El) on a lateral side of the patient femur between the patient femur and patient tibia, with the knee joint in extension. The method also includes automatically setting distal femur resection amounts for the planned distal femur resection. The distal femur resection amounts include a medial distal femoral resection amount and a lateral distal femoral resection amount. The setting the distal femur resection amounts automatically adjusts the medial distal femoral resection amount and lateral distal femoral resection amount such that Em or El is within a threshold of a target. The method additionally includes planning a femoral component rotation for a planned femoral component to be placed on the patient femur. Planning the femoral component rotation automatically plans a rotation amount that provides a desired flexion gap relation between a medial flexion gap (Fm) on the medial side of the patient femur between the patient femur and patient tibia, and a lateral flexion gap (Fl) on the lateral side of the patient femur between the patient femur and patient tibia, with the knee joint in flexion. Further, the method includes selecting a femoral component size for the planned femoral component to be placed on the patient femur. The selecting the femoral component size includes, using a current size of the planned femoral component, setting an angular position of the planned femoral component based on at least one of flexing or extending the planned femoral component to provide a desired relation as between Fm and Em. The selecting the femoral component size also includes determining whether to resize the planned femoral component based on determining whether setting the angular position exceeds a flexion threshold or exceeds an extension threshold. The method also includes determining whether Em and El remain in the desired extension gap relation, and performing processing based on the determining whether EM and El remain in the desired extension gap relation.

In another embodiment, a computer program product is provided for intraoperative surgical resection planning of a surgery to a knee joint of a patient, the knee joint including a patient femur and patient tibia. The computer program product includes a computer readable storage medium storing instructions for execution to perform a method. The method includes setting a cutting angle for a planned distal femur resection of the patient femur that provides a desired extension gap relation between a medial extension gap (Em) on a medial side of the patient femur between the patient femur and patient tibia and a lateral extension gap (El) on a lateral side of the patient femur between the patient femur and patient tibia, with the knee joint in extension. The method also includes automatically setting distal femur resection amounts for the planned distal femur resection. The distal femur resection amounts include a medial distal femoral resection amount and a lateral distal femoral resection amount. The setting the distal femur resection amounts automatically adjusts the medial distal femoral resection amount and lateral distal femoral resection amount such that Em or El is within a threshold of a target. The method additionally includes planning a femoral component rotation for a planned femoral component to be placed on the patient femur. Planning the femoral component rotation automatically plans a rotation amount that provides a desired flexion gap relation between a medial flexion gap (Fm) on the medial side of the patient femur between the patient femur and patient tibia, and a lateral flexion gap (Fl) on the lateral side of the patient femur between the patient femur and patient tibia, with the knee joint in flexion. Further, the method includes selecting a femoral component size for the planned femoral component to be placed on the patient femur. The selecting the femoral component size includes, using a current size of the planned femoral component, setting an angular position of the planned femoral component based on at least one of flexing or extending the planned femoral component to provide a desired relation as between Fm and Em. The selecting the femoral component size also includes determining whether to resize the planned femoral component based on determining whether setting the angular position exceeds a flexion threshold or exceeds an extension threshold. The method also includes determining whether Em and El remain in the desired extension gap relation, and performing processing based on the determining whether EM and El remain in the desired extension gap relation.

In one or more aspects, the desired extension gap relation is a difference of zero between Em and El.

In one or more aspects, setting the distal femur resection amounts adjusts the medial distal femoral resection amount and lateral distal femoral resection amount such that Em is equal to the target.

In one or more aspects, the desired relation as between Fm and Em is a difference of zero between Fm and Em. In one or more aspects, the at least one of the flexing or the extending the planned femoral component is anchored from an anterior portion of the patient femur.

In one or more aspects, targets for Em, El, Fm, and Fl are set based on user input.

In one or more aspects, based on the angular position being within the flexion threshold and being within the extension threshold, the determining whether to resize the planned femoral component determines not to resize the planned femoral component and the selecting the femoral component size selects the current size to be the selected femoral component size.

In one or more aspects, based on the angular position exceeding the flexion threshold or exceeding the extension threshold, the determining whether to resize the planned femoral component determines to resize the planned femoral component and the selecting the femoral component size further includes resizing the planned femoral component. The resizing increases the current size of the femoral component to be a larger component size based on the angular position exceeding the flexion threshold, or decreases the current size of the femoral component to be a smaller component size based on the angular position exceeding the extension threshold. The selectin also includes, based on the resizing, repeating, one or more times: the setting the angular position of the planned femoral component, and the determining whether to resize the planned femoral component. Based on the repeating, the angular position is within the flexion threshold and is within the extension threshold, the determining whether to resize the planned femoral component determines not to resize the planned femoral component, and the selecting the femoral component size selects the current size to be the selected femoral component size.

In one or more aspects, based on determining that Em and El remain in the desired extension gap relation, the performing processing includes providing parameters of the planned distal femur resection to a device, the parameters based on the set cutting angle, distal femur resection amounts, planned femoral component rotation, and angular position, and the selected femoral component size. In one or more aspects, the device includes a display, and the method further includes building and providing a graphical user interface displaying the provided parameters.

In one or more aspects, based on determining that Em and El do not remain in the desired extension gap relation, the performing processing includes repeating, one or more times, the setting the cutting angle, the automatically setting the distal femur resection amounts, the planning the femoral component rotation, the selecting the femoral component size, and the determining whether Em and El remain in the desired extension gap relation.

In another embodiment, a computer system is provided for intraoperative surgical resection planning of a surgery to a knee joint of a patient, the knee joint including a patient femur and patient tibia. The computer system includes a display device, a memory, and a processor in communication with the memory. The computer system is configured to perform a method that includes setting a cutting angle for a planned distal femur resection of the patient femur that provides a desired extension gap relation between a medial extension gap (Em) on a medial side of the patient femur between the patient femur and patient tibia and a lateral extension gap (El) on a lateral side of the patient femur between the patient femur and patient tibia, with the knee joint in extension. The method also includes automatically setting distal femur resection amounts for the planned distal femur resection. The distal femur resection amounts include a medial distal femoral resection amount and a lateral distal femoral resection amount. The setting the distal femur resection amounts automatically adjusts the medial distal femoral resection amount and lateral distal femoral resection amount such that Em or El is within a threshold of a target. The method additionally includes planning a femoral component rotation for a planned femoral component to be placed on the patient femur. Planning the femoral component rotation automatically plans a rotation amount that provides a desired flexion gap relation between a medial flexion gap (Fm) on the medial side of the patient femur between the patient femur and patient tibia, and a lateral flexion gap (Fl) on the lateral side of the patient femur between the patient femur and patient tibia, with the knee joint in flexion. Further, the method includes selecting a femoral component size for the planned femoral component to be placed on the patient femur. The selecting the femoral component size includes, using a current size of the planned femoral component, setting an angular position of the planned femoral component based on at least one of flexing or extending the planned femoral component to provide a desired relation as between Fm and Em. The selecting the femoral component size also includes determining whether to resize the planned femoral component based on determining whether setting the angular position exceeds a flexion threshold or exceeds an extension threshold. The method also includes determining whether Em and El remain in the desired extension gap relation. Additionally, the method includes performing processing based on the determining whether EM and El remain in the desired extension gap relation. Based on determining that Em and El remain in the desired extension gap relation, the performing processing includes providing parameters of the planned distal femur resection to a device, the parameters based on the set cutting angle, distal femur resection amounts, planned femoral component rotation, and angular position, and the selected femoral component size. In addition, the method includes building and providing a graphical user interface displaying the provided parameters.

Additional features and advantages are realized through the techniques described herein. Other embodiments and aspects are described in detail herein and may be considered a part of the claimed aspects.

### BRIEF DESCRIPTION OF THE DRAWINGS

Aspects described herein are particularly pointed out and distinctly claimed as examples in the claims at the conclusion of the specification. The foregoing and other objects, features, and advantages of the disclosure are apparent from the following detailed description taken in conjunction with the accompanying drawings in which:

FIG. 1 depicts an example anatomic tibial resection workflow in accordance with aspects described herein;

FIG. 2 depicts an example schematic representation of tibial cartilage wear;

FIGS. 3A-3B depicts an example process to adjust a femoral component position to balance to a soft tissue target, in accordance with aspects described herein;

FIG. 4 depicts an example balance graph;

FIGS. 5A-5B depict example processes for intraoperative surgical resection planning of a surgery to a knee joint of a patient, in accordance with aspects described herein; and

FIG. 6 depicts an example computer system to perform aspects described herein.

### DETAILED DESCRIPTION

Aspects described herein provide an intraoperative surgical resection planning assistant, for instance one implemented as software, for use in interoperative planning of a robotic-assisted surgical procedure. In some aspects, an intraoperative algorithm is provided in software to automate a process of identifying and achieving optimal targets in robotic-assisted joint replacement surgeries. More specifically, aspects described herein are presented in the context of a knee arthroplasty by way of example, though it should be understood that aspects may be applied to other types of robotic-assisted joint replacement surgeries.

Using intraoperatively generated/obtained patient-specific data, and requiring no pre-operative imaging or planning, aspects can algorithmically achieve balanced knee alignment and tension via the femoral resection aspect of a knee arthroplasty (by way of example) to provide a results-driven outcome. Aspects can correct varying degrees and severity of knee deformity, allowing for consistent and reproducible post-operative outcomes. Aspects may be implant-agnostic and robotic-system-agnostic, relying on intraoperative data collection of a patient's knee balance graph/tension/deformity. Aspects can extend beyond total knee arthroplasty into partial knee replacement aspects (as an example), as well.

Auto-solve aspects described herein can greatly improve efficiency, accuracy, and accessibility in this domain. This will lead to more consistent and reproducible surgical outcomes, and reduce the barriers to adopting data-driven approaches in surgery.

As noted, there are several problems associated with adoption of robotic-assisted-surgical joint replacement technology. For clinical outcomes, there is no current methodology to ensure consistent and optimal results in every surgery, every time. Moreover, current approaches require dedicated personnel to manually manipulate the robotic software, which increases cost. Because there is not currently an automated process, specialized training and education is required for surgeons, clinical staff, and industry representatives to understand the target parameters and operational methodology. In addition to the above, there is often a general resistance to adoption; some surgeons are hesitant to adopt robotic technology due to a preference for familiar, traditional methods. Ambiguity or uncertainty in intraoperative decision-making can lead to longer surgery times, contributing to further resistance, i.e., increased financial and clinical resistance.

Aspects address these and other problems. From a clinical outcome standpoint, processing described herein uses an algorithm that synchronizes with intraoperative data to calculate optimized targets in real-time to consistently meet pre-operative expectations and deliver superior clinical outcomes. Automation can lead to substantial cost savings by reducing the need for manual labor for both care facilities and industry. Automated processes in accordance with embodiments herein can also reduce or eliminate the need for training on the relevant segment(s) of the surgical process. Additionally, removing the uncertainty in identifying optimal targets, as well as how to easily achieve them, can reduce operating room times, provide clinician confidence, and encourage more widespread adoption of robotic-assisted surgery.

Software incorporating aspects described herein can help simplify the surgical process by providing a single surgical solution (e.g., a best or optimal solution) based on clinical data to streamline the targeting process to balance to a soft tissue target. Software-driven targets, for instance patent-specific targets, can be built on a foundation of proven and successful clinical results to be used on patients presenting with variable anatomic factors, delivering improved outcomes for all patients. The process can provide consistent results that can be employed across cases, reducing the need for implant vendor/company representatives to be present. Conventionally one or two such representatives are present for surgery. The software can accelerate the learning curve and aid in education for clinicians and vendor representatives.

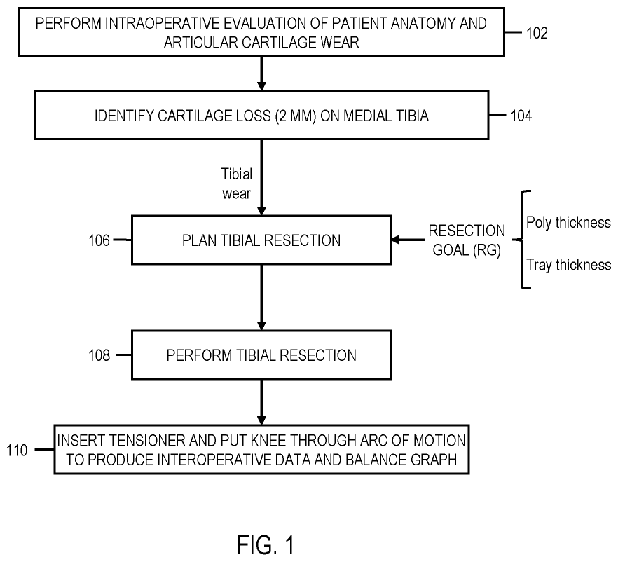

FIG. 1 depicts an example anatomic tibial resection workflow in accordance with aspects described herein. The tibia resection may be set based on an intraoperative evaluation of patient anatomy. The workflow of FIG. 1 is presented in the context of a tibia resection for a patient with mild varus and medial wear on the tibia and femur articular surfaces. Aspects of the workflow could be performed manually (e.g., by a surgeon/user), automatically by a computer system, or a combination of both.

Referring to FIG. 1, the process includes an intraoperative evaluation (102) of patient anatomy and articular cartilage wear. For instance, tibial registration using a robotic wand/probe instrument is used to determine tibial wear (TW). Tibial wear is, in this example, a composite of a medial tibial wear (mTW) and a lateral tibial wear (lTW).

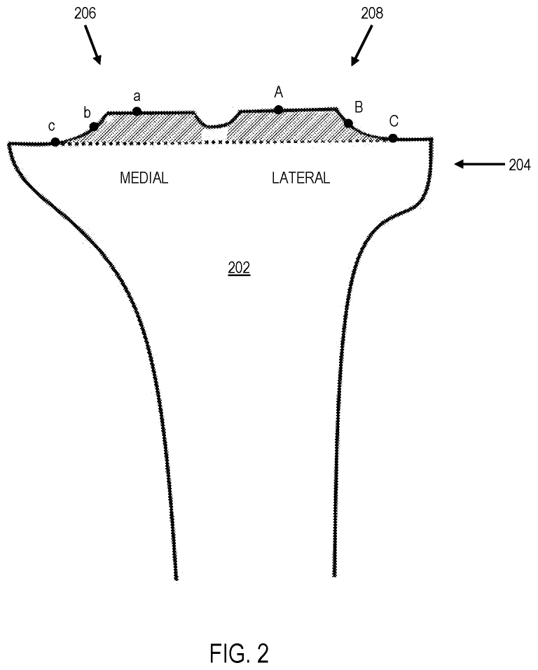

For context, FIG. 2 depicts an example schematic representation of tibial cartilage wear. FIG. 2 depicts a proximal portion 204 of a patient tibia 202. Medial tibial wear (mTW) refers to tibial wear (if any) on the medial side 206 of the tibia. Three levels of medial wear are depicted in this example at points a=normal, b=1 millimeter (mm) cartilage wear, and c=2 mm cartilage wear, corresponding to a “tidemark”, referring to the point where cartilage transitions to bone. Similarly, lateral tibial wear (lTW) refers to tibial wear (if any) on the lateral side 208 of the tibia. Three levels of lateral wear are depicted in this example at points A=normal, B=1 mm cartilage wear, and C=2 mm cartilage wear.

In examples, the surgeon performs tibial registration and determines TW based on anatomic tibial appearance, recorded as mTW and lTW. Possible values discussed above for each of mTW and lTW are 0 mm, 1 mm, and 2 mm of wear, but these are used by way of example only and not limitation. In this example (and referring back to the example workflow of FIG. 1) the registration identifies (104) 2 mm of medial tibial wear and 0 mm of lateral tibial wear.

The TW, including the mTW and lTW components, are parameters used in this example in planning (106) the tibial resection to be performed. Another parameter is the desired thickness of the polyethylene (“poly”) insert as the bearing surface for the knee joint. This may be set/selected by a surgeon or could be set/determined automatically, if desired, and therefore may be upsized or downsized. By way of non-limiting example, a surgeon sets this to be 6 mm. In other examples, a default is used initially that is subject to surgeon modification.

Another parameter is the resection goal (RG). The resection goal may be determined as the sum of the poly thickness and the thickness of the tibial tray used in the surgery. The tibial tray thickness can vary depending on any of various factors. By way of non-limiting example, the tibial tray thickness is 4 mm. Therefore, in this case, RG=6 mm+4 mm=10 mm.

Based on the RG, the planning (106) of the tibial resection determines a medial resection goal (mRG) and a lateral resection goal (lRG). For instance, the medial resection goal (mRG) is determined as the difference between the resection goal (RG) and the medial tibial wear (mTW), and the lateral resection goal (lRG) is determined as the difference between the resection goal (RG) and the lateral tibial wear (lTW). In this example mRG=10 mm−2 mm=8 mm and lRG=10 mm−0 mm=10 mm.

A tibial difference (TD) is the difference in wear between the medial and lateral sides, and informs whether the patient exhibits a varus or valgus condition. If TD=lRG−mRG, then a positive TD reflects a varus condition and a negative TD reflects a valgus condition (TD=0 reflects a neutral alignment). Here, TD=2 mm, reflecting a varus condition. The planned tibial resection can account for any varus/valgus condition, for instance by adjusting so that the difference in the planned medial tibial resection (mTR) on the medial tibia and the planned lateral tibial resection (lTR) on the lateral tibia is the same as the TD (2 mm in this example), meaning a greater amount of tibial wear will result in a lesser amount of resection. In other words, software can automatically adjust the varus/valgus alignment until lTR−mTR=TD. Additionally, the software can adjust the planned resection proximal/distally until the medial resection goal (mRG) equals the medial tibial resection (mTR) and the lateral resection goal (lRG) equals the lateral tibial resection (lTR). In this example, and using the example numbers above, the tibial wear difference of 2 mm informs that the tibial resection should be +2 mm laterally than medially, and (based on the resection goals of 8 mm and 10 mm) should be 8 mm medially and 10 mm laterally. Accordingly, lTR and mTR can be set to be equal to lRG and mRG, respectively.

In one particular example of tibial resection planning, after identifying the medial and lateral tibial wears, a varus/valgus resection adjustment is made to 7 mm medially (mRG=7) and 9 mm laterally (lRG=9) based on a default or other preset Poly thickness (say, 5 mm) and tray thickness (say, 4 mm). The planned resection can then be adjusted distally/proximally as appropriate to accommodate any modifications desired, for instance modifications from surgeon input. For example, the surgeon could adjust the poly insert size to 6 mm from the default/preset 5 mm, in which case the resection would be moved distally 1 mm so that the resection is 8 mm medially and 10 mm laterally to accommodate the 6 mm poly insert indicated by the surgeon. In this manner, the RG values can be set initially based on poly and tray size (in this example), and if the surgeon or other user edits, e.g., component thickness, then the number(s) can adjust dynamically. Different companies might use different values/sizes for poly, tray, and femur implants, in which case modifications to any of these parameters might affect the RG numbers.

Continuing with FIG. 1, the surgeon performs (108) the tibial resection as planned according to the above. This aspect can incorporate additional actions, for instance removal of the meniscus, osteophytes, and loose bodies. The workflow can also include inserting (110) a device, such as a tensioner, and putting the knee through an arc of motion to produce intraoperative soft tissue tension data and a balance graph. In examples, the arc of motion is from approximately 0 degrees-full extension-through approximately 90 degrees of flexion (in this context, ‘approximately’ means within a specified or selected number of degrees, for instance 4 degrees). However, certain factors such as patient characteristics, for instance an arthritis condition, might not allow full extension (flexion contracture). As such, some, and potentially many patients, may only get to within some (e.g., 5 to 10, or less) degrees of full extension. Therefore, the arc of motion through which the knee could be put may be through some range other than 0 to 90 degrees, particularly in situations where 0 degrees extension is unobtainable. In these situations, the range could be between patient-specific maximums, for instance from 90 degrees to some terminal extension other than 0 degrees.

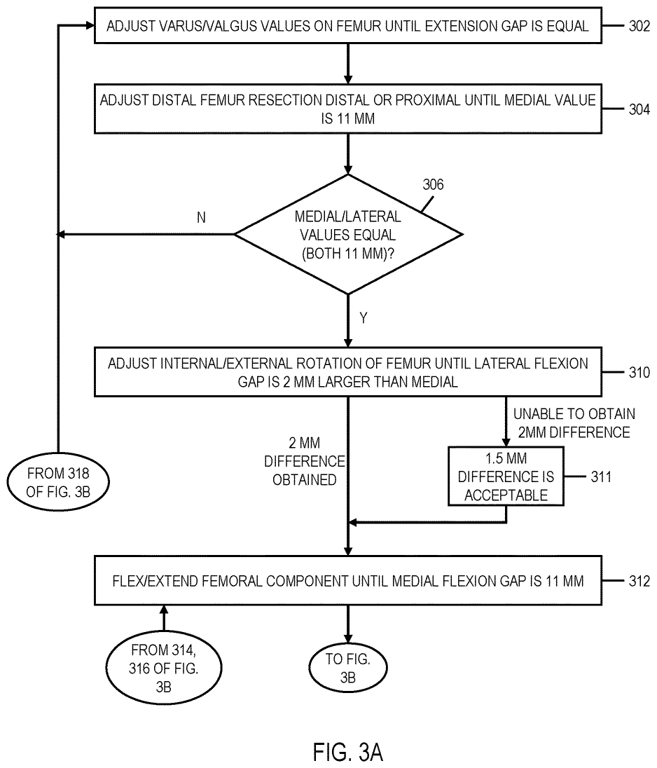

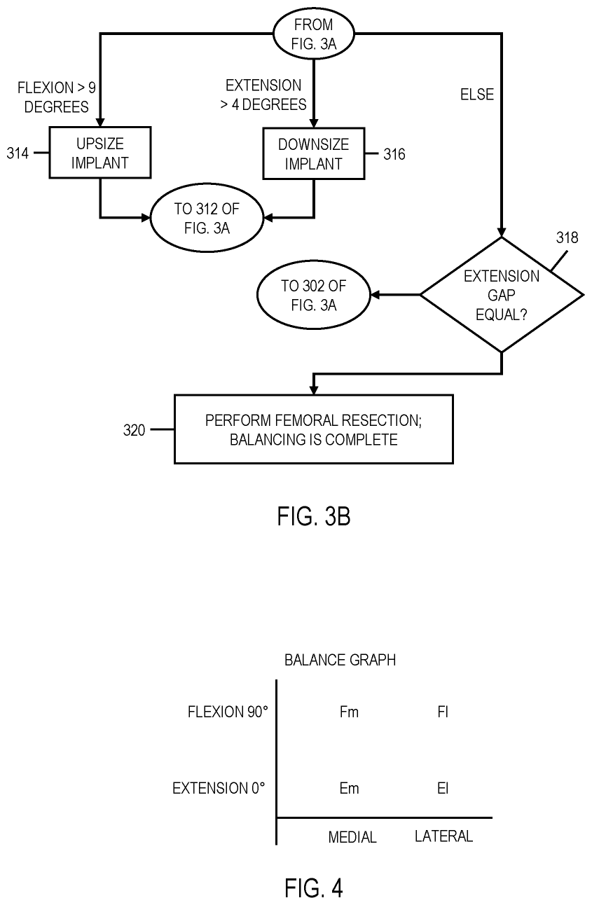

FIGS. 3A-3B depict an example process to adjust a femoral component (implant) position to balance to a soft tissue target, in accordance with aspects described herein. The process of FIGS. 3A-3B could immediately follow from the workflow of FIG. 1, for instance. Aspects of FIGS. 3A-3B could be performed manually (e.g., by a surgeon/user), automatically by a computer system, or a combination of both as part of establishing a plan for a femoral resection.

Three workflows are involved and labeled I, II, and III, with corresponding components as follows:
Ia=femoral varus/valgus;
Ib=femoral proximal/distal;
II=femoral internal external rotation;
IIIa=femoral flexion/extension; and
IIIb=Femoral Component Upsize/downsize.

With respect to IIIb, changing the component size can help manage the flexion space, which could be useful in situations when optimal target(s) cannot otherwise be achieved through flexion. An example of this is upsizing from a size 5 to size 6 femoral component. This upsizing adds component thickness to the flexion space, thereby tightening (or closing) the flexion space. In some example implant offerings, upsizing by 1 size adds 3 mm of component thickness. The opposite happens when downsizing the femoral component. Upsizing the femoral component is something that might regularly be done.

Beginning with component Ia, the process performs (302) a varus/valgus adjustment to properties of the distal femur cut until, e.g., extension gaps Em and El are equal, where Em and El refer to the medial and lateral extension gaps (at full or terminal extension, such as 0 degrees), respectively (refer to FIG. 4 depicting an example balance graph with extension gaps (Em and El) and flexion gaps (Fm and Fl), for flexion 90 degrees and extension 0 degrees, respectively). This adjustment sets/adjusts the distal femur resection levels/amounts for the medial and lateral distal femur (i.e., medial distal femur resection amount and lateral distal femur resection amount), doing so until Em and El are in a desired relation, such as equality to result in an equal extension space gap. For instance, this adjustment can determine these levels/amounts directly to initialize them, or can work against default or initially selected distal femur resection levels/amounts and adjust them to the desired levels/amounts. In some situations, it may not be able to achieve equality as between Em and El, in which case it may be acceptable to proceed so long as they are substantially equal, e.g., within some range considered to be substantially close (such as 0.5 mm, as an example) of each other.

The process then, for component Ib, performs (304) a distal femoral resection adjustment (distal or proximal) to adjust both the medial and lateral distal femoral resection amounts distally or proximally until at least one of the resulting equal extension gaps (Em, El) is/are equal (or substantially equal) to a target. The target could be the resection goal (RG from FIG. 1) itself or could be RG plus a resection distance adjustment, such as 1 or 2 mm, as examples. In some examples, the medial value (i.e., Em) is used as the target because natural kinematics of the knee pivot around the medial condyle of the femur. By way of example and not limitation, the target may be 11 mm as shown in FIG. 3A. The target may be set/selected based on surgeon preference of optimal balance and the resection distance adjustment may be adjusted if desired. In this example, a 1 mm distance adjustment is used as ‘laxity’ that is purposefully built into the system for knee balance. In other situations, a laxity of 0 mm or 2 mm (as examples) are used. Thus, this may be established as a parameter that could be manually (e.g., by a surgeon or other user) and/or automatically (e.g., by a process) selected/adjusted to a desired balance.

The process determines (306) whether the medial and lateral extension gaps (Em, El) (which were initially—at 302—attempted to be made equal (or sufficiently close) remain equal (or sufficiently close), e.g., whether both are 11 mm in this example. Performance of 304 could result in the medial and lateral extension gaps (Em, El) no longer being equal or sufficiently close, for example due to software limitations, specific patient anatomical characteristics, or other reasons. In the case that they are no longer equal or sufficiently close (306, No), the process returns to 302 to make them equal (or sufficiently close) and proceeds.

After adjusting the medial and lateral resection amounts in accordance with 302 and 304 to result in equal extension gaps (or gaps as close as possible after the threshold number of iterations) (306, Y), the process performs (310) an internal/external rotation adjustment until the difference between the medial flexion gap (Fm) at 90 degrees and the lateral flexion gap (Fl) at 90 degrees is a set/selected distance, for instance 2 mm in this example, though it could be any desired, settable target, other examples of which are 0, 1, 3, among others. This distance can also be established as a parameter that could be manually (e.g., by a surgeon or other user) and/or automatically (e.g., by a process) selected/adjusted to a desired balance. This aspect rotates the femoral implant on the end of the patient femur; by rotating, the software determines the gaps based on the implant position on the resected femur. Thus, the adjustment here to modify parameters of resection plans to result in the desired gaps results in the lateral flexion gap Fl being 2 mm (in this example) larger than the medial flexion gap Fm. In some examples, the adjustment need not reach 2 mm but could instead be within some range of this target. In these aspects, parameters, which may be displayed on a user interface, are changed to change the way the femur is resected, and the values in the balancing solution is the result of those resections plus placing the replacement (implant) on the end of the femur.

If the target distance (e.g. 2 mm) was achieved, or if unable to obtain the target difference but able to reach another acceptable difference (311), the process proceeds, for component IIIa, to adjust (flex/extend) (312) the femoral component until the medial flexion gap Fm is equal to the medial extension gap Em (11 mm in this example). Flexing and extending in this context is like ‘tipping’ the implant forward and backward as a way of simulating what would happen with the implant on and making resection angle changes by doing so. This tipping can be anchored from the anterior femur or posterior femur, as examples. Using an anterior anchor point can help protect from cutting into the anterior femur bone and “notching” it. Thus, an anterior anchor point can be used to limit notching.

If the resulting value here becomes more than a first threshold (like 9 degrees flexion) or more than a second threshold (like 3 degrees extension), then implant size is changed. In this manner, the 9 degrees flexion and 3 degrees extension thresholds are example guardrails to not exceed. 9 degrees flexion and 3 degrees extension are used as examples only; there may be different desired thresholds at which to trigger an implant size change.

Thus, the process proceeds to FIG. 3B. If femoral flexion is required greater than some desired first threshold (9 degrees in this example), then the process proceeds to 314 to upsize the femur implant size and return to step 312 to adjust flexion space until Fm=Em. Alternatively, if femoral extension is required to greater than some desired second threshold (neutral, i.e., 0 degrees in the example of FIG. 3B), then the femoral component is downsized at 316 and the process returns to step 312 to adjust the flexion space until Fm=Em. As noted, these parameters could vary. For instance, based on the particular type of femoral component being used (e.g., cruciate retaining CR vs. posterior stabilize PS), the threshold to adjusting the femoral component position/size changes. For example, due to the lack of cruciate ligaments and the presence of a more constraining implant with a post sticking up from the tibia, PS implants generally cannot be flexed as aggressively as CR implants. Thus, the process could determine to upsize the implant at flexion 9 degrees for CR implants but upsize at flexion 5 degrees for PS implants. In some examples, extension greater than 0 degrees will result in the process determining to downsize the femoral component for both CR and PS implant types. In any case, after a size change, the process returns to 312 and adjusts flexion/extension space until Fm=Em.

The process eventually, after any necessary femoral flexion/extension and component size adjustment to comport with the surgeon's boundaries (such as 9 degrees flexion and 3 degrees extension, as examples), thereby proceeding from to 318, inquires at 318 whether the extension gap is equal, i.e., whether Em=El. If not, the process returns to 302. In this regard, flexing or extending the femoral component could potentially change the geometry of the implant in extension, and therefore require the extension gap, which was initially equal, to be adjusted to result in equality medially and laterally. By returning to 302 for continued processing, the progression I, II, III is repeated, and breaks when the soft tissue solves to Fm=Em=El=Fl+2 mm (in this example), where Fm=RG+1 mm (in this example).

In this component flow, aspects I and II are to be completed before aspect III, but it is noted that aspect II could be completed before aspect I (Ia, Ib), if desired. In other words, the internal/external femoral rotation to achieve lateral flexion gap of, e.g., 2 mm larger than medial flexion gap (aspects 310, 311) could be performed before the varus/valgus and distal femur resection adjustments (302 to 304) if desired, prior to proceeding to the femoral flexion/extension aspects (312-316).

If instead inquiry 318 is answered in the positive (extension gap equal), then the process proceeds with performance (320) of the femoral resection, as balancing is complete.

In an alternative embodiment, a tensioner step (e.g., 110) is omitted. The surgeon exposes the knee, clears any osteophytes or loose bodies, then puts the native joint through range of motion. Stress is applied to the knee through the range of motion and a balance graph is obtained based on that. A process as discussed above can then automatically solve the knee balance as discussed above, omitting the tensioner step.

Aspects described herein can be useful in clinical and educational settings. Software implementing features described herein can be integrated into robotic surgery applications for performing robotic surgeries in hospitals and ambulatory surgery center operating rooms.

Accordingly, aspects provide a goal-based and data-driven, targeted solution. In contrast, other approaches might place a heavy emphasis on variable patient data that includes pre-operative non-orthopedic factors (demographics, mental disposition), pre-operative orthopedic factors (BMI, deformity, imaging data), and post-operative data, and essentially captures any possible variables that can be considered, without emphasizing a data-driven target per se, and producing a ranked-order list of possible solutions based on varying surgeon preferences but with no predetermined optimal goal. This is distinguished from aspects described herein in which raw data and balance graph information is used to provide a definitive answer in terms of femoral component position to balance to a soft tissue target (an example of which from above provides 11 mm, 11 mm, 11 mm, 13 mm, which may be determined based on surgeon specificity). In other words, operator selection may be minimized in accordance with aspects herein, which provides simplicity, efficiency, and a removal of the need of resources as discussed above (for instance, in the form of a company representative or additional operating room/team staff member). Automation provided by aspects described herein can also minimize or remove the need to train the company representative and surgeon on the intricacies of surgical robot operation to properly execute the surgical procedure (the learning curve of which may be steep). Such complexity can be a barrier of entry to adopting robotic technology as discussed above. Additionally, aspects described herein can provide a near-instantaneous, single solution without relying on, or provision of, real-time changes; while the surgeon or other user could restart the process and put the patient knee through a new arc of motion, the solver could provide another instantaneous single solution, rather than a real-time adjustment to the previously-determined solution. Further, approaches discussed herein can work around or avoid soft tissue releases, which could sometimes result to compensate for stiffness or other issues.

One or more embodiments described herein may be incorporated in, performed by, and/or used by one or more computer systems, such as one or more computer systems that are incorporated into and/or in communication with an orthopedic surgical robot system. Processes described herein, and/or aspects thereof, may be performed singly or collectively by one or more such computer systems. A computer system may also be referred to herein as a data processing device/system, computing device/system/node, or simply a computer. The computer system may be based on one or more of various system architectures and/or instruction set architectures.

Software executing on a computer system can build and provide (e.g., for graphical display on a display device) a graphical user interface (GUI). The software could be software that performs processes described herein. A GUI includes graphical elements, such as fields, buttons, sliders, toggles, radio selections, images, text values, and others. Some such graphical elements may be interactive in the sense that a user, such as a surgeon or other user of the software, can interact with the elements to set, adjust, select, specify, define, indicate, or the like, desired settings, targets, goals, or any other parameters for processing described herein (such as the surgeon setting the desired soft tissue balance target as part of the interactive balance graph target). Examples of such parameters are those discussed herein related to knee arthroplasty or other joint replacement procedures. In some examples, each such attribute may have a corresponding graphical interface element with which the user might interact to set, adjust, select, specify, etc. a value for that attribute. In this manner, aspects can build, provide and/or display a GUI with elements corresponding to processing parameters, as well as elements to display or otherwise convey results of such processing, for instance values related to balance of a knee joint, as an example, or other outputs of the processing.

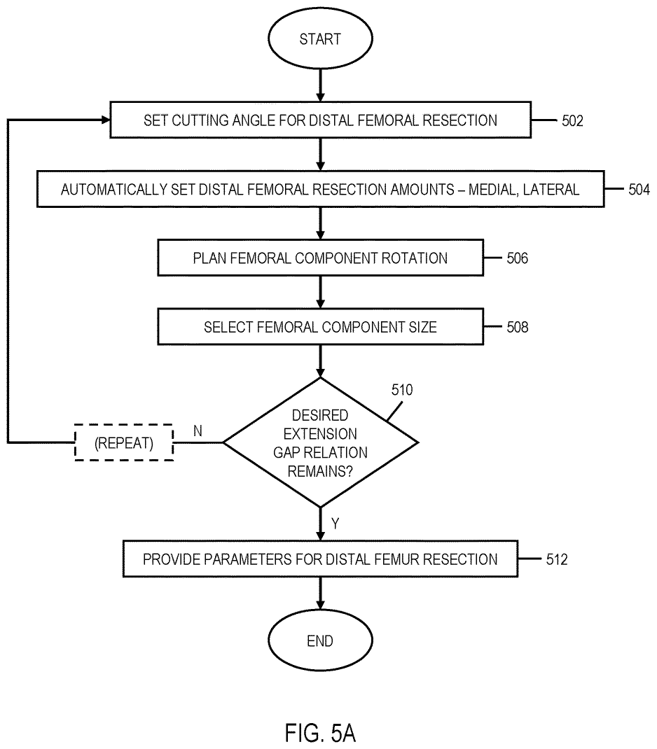

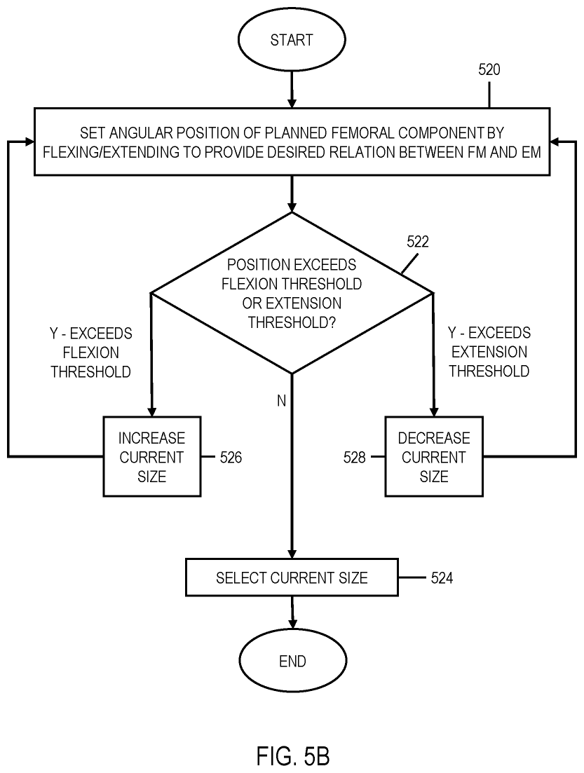

FIGS. 5A-5B depict example processes for intraoperative surgical resection planning of a surgery to a knee joint of a patient, in accordance with aspects described herein. This can be used to achieve balancing to a soft tissue target, for instance. The planning may be performed automatically by software executing on a computer system, and based on intraoperative collection of soft tissue tension data of the patient's knee. Thus, the process may be performed by software as executed, in one or more examples, by a processor or processing circuitry of one or more computers/computer systems, such as those described herein.

Referring initially to FIG. 5A, the process includes setting (502) a cutting angle for a planned distal femur resection of the patient's femur. The setting provides a desired extension gap relation between (i) a medial extension gap (Em) on a medial side of the patient femur between the patient femur and patient tibia and (ii) a lateral extension gap (El) on a lateral side of the patient femur between the patient femur and patient tibia, with the knee joint in extension. The cutting angle can correspond to a varus/valgus adjustment to provide the desired extension gap relation. In particular embodiments, the desired extension gap relation is a difference of zero between Em and El, i.e., Em=El. Alternatively, the desired relation could be that the difference between Em and El is within some range, for instance within a range of 0 millimeters to 0.5 millimeters. The desired relation could be a parameter that is set manually by a user or that is selected automatically, for example.

The process of FIG. 5A proceeds by automatically setting (504) distal femur resection amounts for the planned distal femur resection. The distal femur resection amounts include a medial distal femoral resection amount and a lateral distal femoral resection amount. The setting of the distal femur resection amounts automatically adjusts the medial distal femoral resection amount and lateral distal femoral resection amount such that Em or El is within a threshold of a target. In this aspect, the processing performed as part of the software sets a proximal-distal placement (corresponding to resection amounts on the medial and lateral side) of a distal femoral cut plane that has the set cutting angle, set from 502, so that Em or El (or potentially both) are within a threshold of a target. In examples, this setting of the distal femur resection amounts adjusts the medial distal femoral resection amount and lateral distal femoral resection amount such that Em is equal to the target. In some examples, the target is a resection goal. In some examples, the target incorporates a laxity adjustment. In some examples, the target is based on a resection goal and a laxity adjustment, for instance it is a sum of the two. The target could be a parameter that is set manually by a user or that is selected automatically, for example.

Continuing with FIG. 5A, the process plans (506) a femoral component rotation for a planned femoral component to be placed on the patient femur. This planning of the femoral component rotation automatically plans a rotation amount that provides a desired flexion gap relation between (i) a medial flexion gap (Fm) on the medial side of the patient femur between the patient femur and patient tibia and (ii) a lateral flexion gap (Fl) on the lateral side of the patient femur between the patient femur and patient tibia, with the knee joint in flexion. Rotation of the femoral component can affect the medial and lateral flexion gaps (Fm, Fl, respectively). This aspect plans a rotation of the femoral component so that the resulting Fm and Fl are in a desired flexion gap relation. In examples, the desired flexion gap relation is that Fl is larger than Fm. In some embodiments, the desired relation is that Fl is larger than Fm by a range, for instance 0 millimeters to 3 millimeters, though other tolerable relations are possible. The desired flexion gap relation could be a parameter that is set manually by a user or that is selected automatically, for example.

The process of FIG. 5A then selects (508) a femoral component size for the planned femoral component to be placed on the patient femur. An example process for selecting the femoral component size is described with reference to FIG. 5B. Planning to this point may have used/assumed a default, preset, or otherwise preconfigured component size, however in some examples it might be desired to resize the femoral component in some instances, as described below with reference to FIG. 5B. The selection at 508 therefore potentially, but not necessarily, resizes the femoral component. In any case, this aspect selects the size of the femoral component with which to further proceed with the planning.

Referring to FIG. 5B to select the femoral component size, the process, using a current size of the planned femoral component-which could initially be the assumed size discussed above-sets (520) an angular position of the planned femoral component based on at least one of flexing or extending the planned femoral component to provide a desired relation as between Fm and Em. In this manner, the planned femoral component is flexed/extended to adjust the component placement in an attempt to achieve the desired relation between Fm and Em. In examples, the desired relation as between Fm and Em is a difference of zero between Fm and Em, i.e., Fm=Em. In some embodiments, the flexing and/or the extending of the planned femoral component is anchored from an anterior portion of the patient femur.

FIG. 5B proceeds by determining whether to resize the planned femoral component based on determining (522) whether the setting of the angular position results in exceeding a flexion threshold or exceeding an extension threshold. In accordance with this aspect, there is a flexion threshold, which is a predefined first number of degrees of flexion for example, and an extension threshold, which is a predefined second number of degrees of extension for example, and the determination whether to resize is based on whether either of those is exceeded-that is, whether in performing the setting 520 the component needed to be flexed to an angular position beyond the flexion threshold or extended to an angular position beyond the extension threshold in an attempt to achieve the desired relation between Fm and Em.

In one case, neither the flexion threshold nor the extension threshold are exceeded by the angular position in attempting to achieve the desired relation. In other words, based on the setting at 522 resulting in a set angular position that is within the flexion threshold and within the extension threshold, it is determined not to resize the planned femoral component (522, N). The current size of the femoral component is deemed the correct size, and the process proceeds to 524 to select the current size to be the selected femoral component size, providing the selected size at 508 of FIG. 5A.

If instead the angular position exceeds the flexion threshold or exceeds the extension threshold, it is determined to resize the planned femoral component. Specifically, if it is determined at 522 that the angular position exceeds the flexion threshold (Y-exceeds flexion threshold), the planned femoral component is resized, specifically is upsized (526) to be a larger component size. If instead it is determined at 522 that the angular position exceeds the extension threshold (Y-exceeds extension threshold), the planned femoral component is resized, specifically is downsized (528) to be a smaller component size. The upsizing or downsizing increases or decreases, as the case may be, the ‘current’ size being used in the planning.

In the case that the current size of the planned femoral component is resized by 526 or 528, the process returns to 520 to repeat the setting the angular position (520) of the planned femoral component which has now been resized such that the ‘current’ size is now bigger or smaller, followed by another determination whether to resize the planned femoral component by determining (522) whether either threshold is exceeded. This repeating may be performed one or more times. Eventually, based on the repeating and potential upsizing or downsizing, the angular position is within the flexion threshold and is within the extension threshold, i.e., it does not exceed either threshold (522, N), and the determination is made that the planned femoral component at the then-current size does not need further resizing. The current size is then selected at 524 to be the selected femoral component size, providing the selected size at 508 of FIG. 5A.

Returning back to FIG. 5A, with the femoral component size selected, the process determines (510) whether Em and El remain in the desired extension gap relation. It is possible based on performing prior aspects of FIG. 5A that the relation between Em and El has changed is no longer in the desired relation. The process will proceed by performing processing based on that determination at 510 as to whether EM and El remain in the desired extension gap relation. For instance, based on determining that Em and El do not remain in the desired extension gap relation (510, N), the performing processing includes returning to 502 to repeat the setting the cutting angle (502), the automatically setting (504) the distal femur resection amounts, the planning (506) the femoral component rotation, the selecting (508) the femoral component size, and the determining (510) whether Em and El remain in the desired extension gap relation. This repetition could be performed one or more times until the desired extension gap remains at 510. When returning to 502, the then-current size of the femoral component may be used in the planning at 502, 504 and 506, with the possibility that the subsequent size selection at 508 will upsize or downsize the component, or will select the then-current size as the size with which to continue.

If instead it is determined at 510 that Em and El remain in the desired extension gap relation, the performing processing includes providing parameters of the planned distal femur resection to a device. The parameters provided can be based on the set cutting angle, distal femur resection amounts, planned femoral component rotation, and angular position, and on the selected femoral component size. In some embodiments, the device is a display device, and the process further includes building and providing a graphical user interface that displays the provided parameters. Additionally or alternatively, the device could be a surgical robot system configured to use the parameters to perform surgical actions, for instance a cutting action to fully or partially automatically perform the distal femur resection.

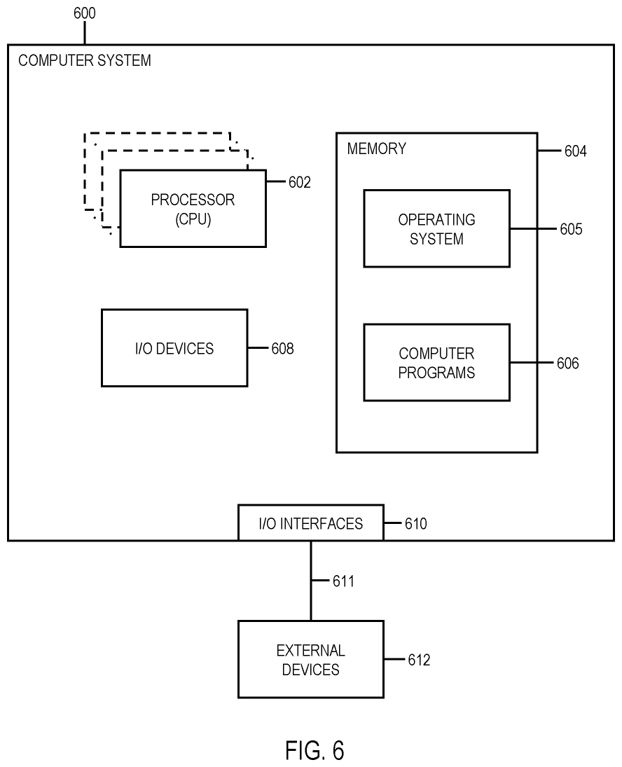

FIG. 6 shows a computer system 600 in communication with external device(s) 612. Computer system 600 includes one or more processor(s) 602, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 602 can also include register(s) to be used by one or more of the functional components. Computer system 600 also includes memory 604, input/output (I/O) devices 608, and I/O interfaces 610, which may be coupled to processor(s) 602 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

Memory 604 can be or include main or system memory (e.g., Random Access Memory) used in the execution of program instructions, storage device(s) such as hard drive(s), flash media, or optical media as examples, and/or cache memory, as examples. Memory 604 can include, for instance, a cache, such as a shared cache, which may be coupled to local caches (examples include L1 cache, L2 cache, etc.) of processor(s) 602. Additionally, memory 604 may be or include at least one computer program product having a set (e.g., at least one) of program modules, instructions, code, or the like that is/are configured to carry out functions of embodiments described herein when executed by one or more processors.

Memory 604 can store an operating system 605 and other computer programs 606, such as one or more computer programs/applications that execute to perform aspects described herein. Specifically, programs/applications can include computer readable program instructions that may be configured to carry out functions of embodiments of aspects described herein.

Examples of I/O devices 608 include but are not limited to microphones, speakers, Global Positioning System (GPS) devices, RGB, IR, spectral, and/or other forms of cameras, lights, accelerometers, gyroscopes, magnetometers, sensor devices configured to sense light, proximity, heart rate, body and/or ambient temperature, blood pressure, and/or skin resistance, registration probes, robotic tools, and activity monitors. An I/O device may be incorporated into the computer system as shown, though in some embodiments an I/O device may be regarded as an external device (612) coupled to the computer system through one or more I/O interfaces 610.

Computer system 600 may communicate with one or more external devices 612 via one or more I/O interfaces 610. Example external devices include a keyboard, a pointing device, a display, and/or any other devices that enable a user to interact with computer system 600. Other example external devices include any device that enables computer system 600 to communicate with one or more other computing systems or peripheral devices such as a printer. A network interface/adapter is an example I/O interface that enables computer system 600 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 610 and external devices 612 can occur across wired and/or wireless communications link(s) 611, such as Ethernet-based wired or wireless connections. Example wireless connections include cellular, Wi-Fi, Bluetooth®, proximity-based, near-field, or other types of wireless connections. More generally, communications link(s) 611 may be any appropriate wireless and/or wired communication link(s) for communicating data.

Particular external device(s) 612 may include one or more data storage devices, which may store one or more programs, one or more computer readable program instructions, and/or data, etc. Computer system 600 may include and/or be coupled to and in communication with (e.g., as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Computer system 600 may be operational with numerous other general purpose or special purpose computing system environments or configurations. Computer system 600 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

Aspects of the present invention may be a system, a method, and/or a computer program product, any of which may be configured to perform or facilitate aspects described herein.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g., instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C#, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

Although various embodiments are described above, these are only examples.

Provided is a small sampling of embodiments of the present disclosure, as described herein:
A1. A computer-implemented method for intraoperative surgical resection planning of a surgery to a knee joint of a patient, the knee joint including a patient femur and patient tibia, the method including: setting a cutting angle for a planned distal femur resection of the patient femur that provides a desired extension gap relation between a medial extension gap (Em) on a medial side of the patient femur between the patient femur and patient tibia and a lateral extension gap (El) on a lateral side of the patient femur between the patient femur and patient tibia, with the knee joint in extension; automatically setting distal femur resection amounts for the planned distal femur resection, the distal femur resection amounts including a medial distal femoral resection amount and a lateral distal femoral resection amount, wherein the setting the distal femur resection amounts automatically adjusts the medial distal femoral resection amount and lateral distal femoral resection amount such that Em or El is within a threshold of a target; planning a femoral component rotation for a planned femoral component to be placed on the patient femur, wherein planning the femoral component rotation automatically plans a rotation amount that provides a desired flexion gap relation between a medial flexion gap (Fm) on the medial side of the patient femur between the patient femur and patient tibia, and a lateral flexion gap (Fl) on the lateral side of the patient femur between the patient femur and patient tibia, with the knee joint in flexion; selecting a femoral component size for the planned femoral component to be placed on the patient femur, wherein the selecting the femoral component size includes: using a current size of the planned femoral component, setting an angular position of the planned femoral component based on at least one of flexing or extending the planned femoral component to provide a desired relation as between Fm and Em; and determining whether to resize the planned femoral component based on determining whether setting the angular position exceeds a flexion threshold or exceeds an extension threshold; determining whether Em and El remain in the desired extension gap relation; and performing processing based on the determining whether EM and El remain in the desired extension gap relation.
A2. The method of A1, wherein the desired extension gap relation is a difference of zero between Em and El.
A3. The method of A1, wherein the desired extension gap relation is a difference between Em and El that is within a range of 0 millimeters to 0.5 millimeters.
A4. The method of A1, wherein the setting the distal femur resection amounts adjusts the medial distal femoral resection amount and lateral distal femoral resection amount such that Em is equal to the target.
A5. The method of A4, wherein the target is based on a resection goal and a laxity adjustment.
A6. The method of A1, wherein the desired flexion gap relation is Fl being larger than Fm.
A7. The method of A6, wherein the desired flexion gap relation is Fl being larger than Fm by 0 millimeters to 3 millimeters.
A8. The method of A1, wherein the desired relation as between Fm and Em is a difference of zero between Fm and Em.
A9. The method of A8, wherein the at least one of the flexing or the extending the planned femoral component is anchored from an anterior portion of the patient femur.
A10. The method of A1, wherein the flexion threshold is a predefined first number of degrees and the extension threshold is a predefined second number of degrees that is less than the first number of degrees.
A11. The method of A1, wherein based on the angular position being within the flexion threshold and being within the extension threshold, the determining whether to resize the planned femoral component determines not to resize the planned femoral component and the selecting the femoral component size selects the current size to be the selected femoral component size.
A12. The method of A1, wherein based on the angular position exceeding the flexion threshold or exceeding the extension threshold, the determining whether to resize the planned femoral component determines to resize the planned femoral component, and wherein the selecting the femoral component size further includes: resizing the planned femoral component, wherein the resizing increases the current size of the femoral component to be a larger component size based on the angular position exceeding the flexion threshold, or decreases the current size of the femoral component to be a smaller component size based on the angular position exceeding the extension threshold; and based on the resizing, repeating, one or more times: the setting the angular position of the planned femoral component, and the determining whether to resize the planned femoral component, wherein, based on the repeating, the angular position is within the flexion threshold and is within the extension threshold, the determining whether to resize the planned femoral component determines not to resize the planned femoral component, and the selecting the femoral component size selects the current size to be the selected femoral component size.
A13. The method of A1, wherein based on determining that Em and El remain in the desired extension gap relation, the performing processing includes providing parameters of the planned distal femur resection to a device, the parameters based on the set cutting angle, distal femur resection amounts, planned femoral component rotation, and angular position, and the selected femoral component size.
A14. The method of A13, wherein the device includes a display, and wherein the method further includes building and providing a graphical user interface displaying the provided parameters.
A15. The method of A13, wherein the device is a surgical robot system.
A16. The method of A1, wherein based on determining that Em and El do not remain in the desired extension gap relation, the performing processing includes repeating, one or more times, the setting the cutting angle, the automatically setting the distal femur resection amounts, the planning the femoral component rotation, the selecting the femoral component size, and the determining whether Em and El remain in the desired extension gap relation.
B1. A computer program product for intraoperative surgical resection planning of a surgery to a knee joint of a patient, the knee joint including a patient femur and patient tibia, the computer program product including: a computer readable storage medium storing instructions for execution to perform a method including: setting a cutting angle for a planned distal femur resection of the patient femur that provides a desired extension gap relation between a medial extension gap (Em) on a medial side of the patient femur between the patient femur and patient tibia and a lateral extension gap (El) on a lateral side of the patient femur between the patient femur and patient tibia, with the knee joint in extension; automatically setting distal femur resection amounts for the planned distal femur resection, the distal femur resection amounts including a medial distal femoral resection amount and a lateral distal femoral resection amount, wherein the setting the distal femur resection amounts automatically adjusts the medial distal femoral resection amount and lateral distal femoral resection amount such that Em or El is within a threshold of a target; planning a femoral component rotation for a planned femoral component to be placed on the patient femur, wherein planning the femoral component rotation automatically plans a rotation amount that provides a desired flexion gap relation between a medial flexion gap (Fm) on the medial side of the patient femur between the patient femur and patient tibia, and a lateral flexion gap (Fl) on the lateral side of the patient femur between the patient femur and patient tibia, with the knee joint in flexion; selecting a femoral component size for the planned femoral component to be placed on the patient femur, wherein the selecting the femoral component size includes: using a current size of the planned femoral component, setting an angular position of the planned femoral component based on at least one of flexing or extending the planned femoral component to provide a desired relation as between Fm and Em; and determining whether to resize the planned femoral component based on determining whether setting the angular position exceeds a flexion threshold or exceeds an extension threshold; determining whether Em and El remain in the desired extension gap relation; and performing processing based on the determining whether EM and El remain in the desired extension gap relation.
B2. The computer program product of B1, wherein the desired extension gap relation is a difference of zero between Em and El.
B3. The computer program product of B1, wherein the desired extension gap relation is a difference between Em and El that is within a range of 0 millimeters to 0.5 millimeters.
B4. The computer program product of B1, wherein the setting the distal femur resection amounts adjusts the medial distal femoral resection amount and lateral distal femoral resection amount such that Em is equal to the target.
B5. The computer program product of B4, wherein the target is based on a resection goal and a laxity adjustment.
B6. The computer program product of B1, wherein the desired flexion gap relation is Fl being larger than Fm.
B7. The computer program product of B6, wherein the desired flexion gap relation is Fl being larger than Fm by 0 millimeters to 3 millimeters.
B8. The computer program product of B1, wherein the desired relation as between Fm and Em is a difference of zero between Fm and Em.
B9. The computer program product of B8, wherein the at least one of the flexing or the extending the planned femoral component is anchored from an anterior portion of the patient femur.
B10. The computer program product of B1, wherein the flexion threshold is a predefined first number of degrees and the extension threshold is a predefined second number of degrees that is less than the first number of degrees.
B11. The computer program product of B1, wherein based on the angular position being within the flexion threshold and being within the extension threshold, the determining whether to resize the planned femoral component determines not to resize the planned femoral component and the selecting the femoral component size selects the current size to be the selected femoral component size.
B12. The computer program product of B1, wherein based on the angular position exceeding the flexion threshold or exceeding the extension threshold, the determining whether to resize the planned femoral component determines to resize the planned femoral component, and wherein the selecting the femoral component size further includes: resizing the planned femoral component, wherein the resizing increases the current size of the femoral component to be a larger component size based on the angular position exceeding the flexion threshold, or decreases the current size of the femoral component to be a smaller component size based on the angular position exceeding the extension threshold; and based on the resizing, repeating, one or more times: the setting the angular position of the planned femoral component, and the determining whether to resize the planned femoral component, wherein, based on the repeating, the angular position is within the flexion threshold and is within the extension threshold, the determining whether to resize the planned femoral component determines not to resize the planned femoral component, and the selecting the femoral component size selects the current size to be the selected femoral component size.
B13. The computer program product of B1, wherein based on determining that Em and El remain in the desired extension gap relation, the performing processing includes providing parameters of the planned distal femur resection to a device, the parameters based on the set cutting angle, distal femur resection amounts, planned femoral component rotation, and angular position, and the selected femoral component size.
B14. The computer program product of B13, wherein the device includes a display, and wherein the method further includes building and providing a graphical user interface displaying the provided parameters.
B15. The computer program product of B13, wherein the device is a surgical robot system.
B16. The computer program product of B1, wherein based on determining that Em and El do not remain in the desired extension gap relation, the performing processing includes repeating, one or more times, the setting the cutting angle, the automatically setting the distal femur resection amounts, the planning the femoral component rotation, the selecting the femoral component size, and the determining whether Em and El remain in the desired extension gap relation.
C1. A computer system for intraoperative surgical resection planning of a surgery to a knee joint of a patient, the knee joint including a patient femur and patient tibia, the computer system including: a memory; and a processor in communication with the memory, wherein the computer system is configured to perform a method including: setting a cutting angle for a planned distal femur resection of the patient femur that provides a desired extension gap relation between a medial extension gap (Em) on a medial side of the patient femur between the patient femur and patient tibia and a lateral extension gap (El) on a lateral side of the patient femur between the patient femur and patient tibia, with the knee joint in extension; automatically setting distal femur resection amounts for the planned distal femur resection, the distal femur resection amounts including a medial distal femoral resection amount and a lateral distal femoral resection amount, wherein the setting the distal femur resection amounts automatically adjusts the medial distal femoral resection amount and lateral distal femoral resection amount such that Em or El is within a threshold of a target; planning a femoral component rotation for a planned femoral component to be placed on the patient femur, wherein planning the femoral component rotation automatically plans a rotation amount that provides a desired flexion gap relation between a medial flexion gap (Fm) on the medial side of the patient femur between the patient femur and patient tibia, and a lateral flexion gap (Fl) on the lateral side of the patient femur between the patient femur and patient tibia, with the knee joint in flexion; selecting a femoral component size for the planned femoral component to be placed on the patient femur, wherein the selecting the femoral component size includes: using a current size of the planned femoral component, setting an angular position of the planned femoral component based on at least one of flexing or extending the planned femoral component to provide a desired relation as between Fm and Em; and determining whether to resize the planned femoral component based on determining whether setting the angular position exceeds a flexion threshold or exceeds an extension threshold; determining whether Em and El remain in the desired extension gap relation; and performing processing based on the determining whether EM and El remain in the desired extension gap relation.
C2. The computer system of C1, wherein the desired extension gap relation is a difference of zero between Em and El.
C3. The computer system of C1, wherein the desired extension gap relation is a difference between Em and El that is within a range of 0 millimeters to 0.5 millimeters.
C4. The computer system of C1, wherein the setting the distal femur resection amounts adjusts the medial distal femoral resection amount and lateral distal femoral resection amount such that Em is equal to the target.
C5. The computer system of C4, wherein the target is based on a resection goal and a laxity adjustment.
C6. The computer system of C1, wherein the desired flexion gap relation is Fl being larger than Fm.
C7. The computer system of C6, wherein the desired flexion gap relation is Fl being larger than Fm by 0 millimeters to 3 millimeters.
C8. The computer system of C1, wherein the desired relation as between Fm and Em is a difference of zero between Fm and Em.
C9. The computer system of C8, wherein the at least one of the flexing or the extending the planned femoral component is anchored from an anterior portion of the patient femur.
C10. The computer system of C1, wherein the flexion threshold is a predefined first number of degrees and the extension threshold is a predefined second number of degrees that is less than the first number of degrees.
C11. The computer system of C1, wherein based on the angular position being within the flexion threshold and being within the extension threshold, the determining whether to resize the planned femoral component determines not to resize the planned femoral component and the selecting the femoral component size selects the current size to be the selected femoral component size.
C12. The computer system of C1, wherein based on the angular position exceeding the flexion threshold or exceeding the extension threshold, the determining whether to resize the planned femoral component determines to resize the planned femoral component, and wherein the selecting the femoral component size further includes: resizing the planned femoral component, wherein the resizing increases the current size of the femoral component to be a larger component size based on the angular position exceeding the flexion threshold, or decreases the current size of the femoral component to be a smaller component size based on the angular position exceeding the extension threshold; and based on the resizing, repeating, one or more times: the setting the angular position of the planned femoral component, and the determining whether to resize the planned femoral component, wherein, based on the repeating, the angular position is within the flexion threshold and is within the extension threshold, the determining whether to resize the planned femoral component determines not to resize the planned femoral component, and the selecting the femoral component size selects the current size to be the selected femoral component size.
C13. The computer system of C1, wherein based on determining that Em and El remain in the desired extension gap relation, the performing processing includes providing parameters of the planned distal femur resection to a device, the parameters based on the set cutting angle, distal femur resection amounts, planned femoral component rotation, and angular position, and the selected femoral component size.
C14. The computer system of C13, wherein the device includes a display, and wherein the method further includes building and providing a graphical user interface displaying the provided parameters.
C15. The computer system of C13, wherein the device is a surgical robot system.
C16. The computer system of C1, wherein based on determining that Em and El do not remain in the desired extension gap relation, the performing processing includes repeating, one or more times, the setting the cutting angle, the automatically setting the distal femur resection amounts, the planning the femoral component rotation, the selecting the femoral component size, and the determining whether Em and El remain in the desired extension gap relation.
D1. A computer system for intraoperative surgical resection planning of a surgery to a knee joint of a patient, the knee joint including a patient femur and patient tibia, the computer system including: a display device; a memory; and a processor in communication with the memory, wherein the computer system is configured to perform a method including: setting a cutting angle for a planned distal femur resection of the patient femur that provides a desired extension gap relation between a medial extension gap (Em) on a medial side of the patient femur between the patient femur and patient tibia and a lateral extension gap (El) on a lateral side of the patient femur between the patient femur and patient tibia, with the knee joint in extension; automatically setting distal femur resection amounts for the planned distal femur resection, the distal femur resection amounts including a medial distal femoral resection amount and a lateral distal femoral resection amount, wherein the setting the distal femur resection amounts automatically adjusts the medial distal femoral resection amount and lateral distal femoral resection amount such that Em or El is within a threshold of a target; planning a femoral component rotation for a planned femoral component to be placed on the patient femur, wherein planning the femoral component rotation automatically plans a rotation amount that provides a desired flexion gap relation between a medial flexion gap (Fm) on the medial side of the patient femur between the patient femur and patient tibia, and a lateral flexion gap (Fl) on the lateral side of the patient femur between the patient femur and patient tibia, with the knee joint in flexion; selecting a femoral component size for the planned femoral component to be placed on the patient femur, wherein the selecting the femoral component size includes: using a current size of the planned femoral component, setting an angular position of the planned femoral component based on at least one of flexing or extending the planned femoral component to provide a desired relation as between Fm and Em; and determining whether to resize the planned femoral component based on determining whether setting the angular position exceeds a flexion threshold or exceeds an extension threshold; determining whether Em and El remain in the desired extension gap relation; performing processing based on the determining whether EM and El remain in the desired extension gap relation, wherein based on determining that Em and El remain in the desired extension gap relation, the performing processing includes providing parameters of the planned distal femur resection to a device, the parameters based on the set cutting angle, distal femur resection amounts, planned femoral component rotation, and angular position, and the selected femoral component size; and building and providing a graphical user interface displaying the provided parameters.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A computer-implemented method for intraoperative surgical resection planning of a surgery to a knee joint of a patient, the knee joint including a patient femur and patient tibia, the method including:
setting a cutting angle for a planned distal femur resection of the patient femur that provides a desired extension gap relation between a medial extension gap (Em) on a medial side of the patient femur between the patient femur and patient tibia and a lateral extension gap (El) on a lateral side of the patient femur between the patient femur and patient tibia, with the knee joint in extension;
automatically setting distal femur resection amounts for the planned distal femur resection, the distal femur resection amounts including a medial distal femoral resection amount and a lateral distal femoral resection amount, wherein the setting the distal femur resection amounts automatically adjusts the medial distal femoral resection amount and lateral distal femoral resection amount such that Em or El is within a threshold of a target;
planning a femoral component rotation for a planned femoral component to be placed on the patient femur, wherein planning the femoral component rotation automatically plans a rotation amount that provides a desired flexion gap relation between a medial flexion gap (Fm) on the medial side of the patient femur between the patient femur and patient tibia, and a lateral flexion gap (Fl) on the lateral side of the patient femur between the patient femur and patient tibia, with the knee joint in flexion;
selecting a femoral component size for the planned femoral component to be placed on the patient femur, wherein the selecting the femoral component size includes:
using a current size of the planned femoral component, setting an angular position of the planned femoral component based on at least one of flexing or extending the planned femoral component to provide a desired relation as between Fm and Em; and
determining whether to resize the planned femoral component based on determining whether setting the angular position exceeds a flexion threshold or exceeds an extension threshold;
determining whether Em and El remain in the desired extension gap relation; and
performing processing based on the determining whether EM and El remain in the desired extension gap relation.

2. The method of claim 1, wherein the desired extension gap relation is a difference of zero between Em and El.

3. The method of claim 1, wherein the setting the distal femur resection amounts adjusts the medial distal femoral resection amount and lateral distal femoral resection amount such that Em is equal to the target.

4. The method of claim 1, wherein the desired relation as between Fm and Em is a difference of zero between Fm and Em.

5. The method of claim 4, wherein the at least one of the flexing or the extending the planned femoral component is anchored from an anterior portion of the patient femur.

6. The method of claim 1, wherein targets for Em, El, Fm, and Fl are set based on user input.

7. The method of claim 1, wherein based on the angular position being within the flexion threshold and being within the extension threshold, the determining whether to resize the planned femoral component determines not to resize the planned femoral component and the selecting the femoral component size selects the current size to be the selected femoral component size.

8. The method of claim 1, wherein based on the angular position exceeding the flexion threshold or exceeding the extension threshold, the determining whether to resize the planned femoral component determines to resize the planned femoral component, and wherein the selecting the femoral component size further includes:
resizing the planned femoral component, wherein the resizing increases the current size of the femoral component to be a larger component size based on the angular position exceeding the flexion threshold, or decreases the current size of the femoral component to be a smaller component size based on the angular position exceeding the extension threshold; and
based on the resizing, repeating, one or more times: the setting the angular position of the planned femoral component, and the determining whether to resize the planned femoral component, wherein, based on the repeating, the angular position is within the flexion threshold and is within the extension threshold, the determining whether to resize the planned femoral component determines not to resize the planned femoral component, and the selecting the femoral component size selects the current size to be the selected femoral component size.

9. The method of claim 1, wherein based on determining that Em and El remain in the desired extension gap relation, the performing processing includes providing parameters of the planned distal femur resection to a device, the parameters based on the set cutting angle, distal femur resection amounts, planned femoral component rotation, and angular position, and the selected femoral component size.

10. The method of claim 9, wherein the device includes a display, and wherein the method further includes building and providing a graphical user interface displaying the provided parameters.

11. The method of claim 1, wherein based on determining that Em and El do not remain in the desired extension gap relation, the performing processing includes repeating, one or more times, the setting the cutting angle, the automatically setting the distal femur resection amounts, the planning the femoral component rotation, the selecting the femoral component size, and the determining whether Em and El remain in the desired extension gap relation.

12. A computer program product for intraoperative surgical resection planning of a surgery to a knee joint of a patient, the knee joint including a patient femur and patient tibia, the computer program product including:
a computer readable storage medium storing instructions for execution to perform a method including:
setting a cutting angle for a planned distal femur resection of the patient femur that provides a desired extension gap relation between a medial extension gap (Em) on a medial side of the patient femur between the patient femur and patient tibia and a lateral extension gap (El) on a lateral side of the patient femur between the patient femur and patient tibia, with the knee joint in extension;
automatically setting distal femur resection amounts for the planned distal femur resection, the distal femur resection amounts including a medial distal femoral resection amount and a lateral distal femoral resection amount, wherein the setting the distal femur resection amounts automatically adjusts the medial distal femoral resection amount and lateral distal femoral resection amount such that Em or El is within a threshold of a target;
planning a femoral component rotation for a planned femoral component to be placed on the patient femur, wherein planning the femoral component rotation automatically plans a rotation amount that provides a desired flexion gap relation between a medial flexion gap (Fm) on the medial side of the patient femur between the patient femur and patient tibia, and a lateral flexion gap (Fl) on the lateral side of the patient femur between the patient femur and patient tibia, with the knee joint in flexion;
selecting a femoral component size for the planned femoral component to be placed on the patient femur, wherein the selecting the femoral component size includes:
using a current size of the planned femoral component, setting an angular position of the planned femoral component based on at least one of flexing or extending the planned femoral component to provide a desired relation as between Fm and Em; and
determining whether to resize the planned femoral component based on determining whether setting the angular position exceeds a flexion threshold or exceeds an extension threshold;
determining whether Em and El remain in the desired extension gap relation; and
performing processing based on the determining whether EM and El remain in the desired extension gap relation.

13. The computer program product of claim 12, wherein the desired extension gap relation is a difference of zero between Em and El.

14. The computer program product of claim 12, wherein the setting the distal femur resection amounts adjusts the medial distal femoral resection amount and lateral distal femoral resection amount such that Em is equal to the target.

15. The computer program product of claim 12, wherein the desired relation as between Fm and Em is a difference of zero between Fm and Em, and wherein the at least one of the flexing or the extending the planned femoral component is anchored from an anterior portion of the patient femur.

16. The computer program product of claim 11, wherein based on the angular position being within the flexion threshold and being within the extension threshold, the determining whether to resize the planned femoral component determines not to resize the planned femoral component and the selecting the femoral component size selects the current size to be the selected femoral component size.

17. The computer program product of claim 11, wherein based on the angular position exceeding the flexion threshold or exceeding the extension threshold, the determining whether to resize the planned femoral component determines to resize the planned femoral component, and wherein the selecting the femoral component size further includes:
resizing the planned femoral component, wherein the resizing increases the current size of the femoral component to be a larger component size based on the angular position exceeding the flexion threshold, or decreases the current size of the femoral component to be a smaller component size based on the angular position exceeding the extension threshold; and
based on the resizing, repeating, one or more times: the setting the angular position of the planned femoral component, and the determining whether to resize the planned femoral component, wherein, based on the repeating, the angular position is within the flexion threshold and is within the extension threshold, the determining whether to resize the planned femoral component determines not to resize the planned femoral component, and the selecting the femoral component size selects the current size to be the selected femoral component size.

18. The computer program product of claim 11, wherein based on determining that Em and El remain in the desired extension gap relation, the performing processing includes providing parameters of the planned distal femur resection to a device, the parameters based on the set cutting angle, distal femur resection amounts, planned femoral component rotation, and angular position, and the selected femoral component size.

19. The computer program product of claim 11, wherein based on determining that Em and El do not remain in the desired extension gap relation, the performing processing includes repeating, one or more times, the setting the cutting angle, the automatically setting the distal femur resection amounts, the planning the femoral component rotation, the selecting the femoral component size, and the determining whether Em and El remain in the desired extension gap relation.

20. A computer system for intraoperative surgical resection planning of a surgery to a knee joint of a patient, the knee joint including a patient femur and patient tibia, the computer system including:
a display device;
a memory; and
a processor in communication with the memory, wherein the computer system is configured to perform a method including:
setting a cutting angle for a planned distal femur resection of the patient femur that provides a desired extension gap relation between a medial extension gap (Em) on a medial side of the patient femur between the patient femur and patient tibia and a lateral extension gap (El) on a lateral side of the patient femur between the patient femur and patient tibia, with the knee joint in extension;
automatically setting distal femur resection amounts for the planned distal femur resection, the distal femur resection amounts including a medial distal femoral resection amount and a lateral distal femoral resection amount, wherein the setting the distal femur resection amounts automatically adjusts the medial distal femoral resection amount and lateral distal femoral resection amount such that Em or El is within a threshold of a target;
planning a femoral component rotation for a planned femoral component to be placed on the patient femur, wherein planning the femoral component rotation automatically plans a rotation amount that provides a desired flexion gap relation between a medial flexion gap (Fm) on the medial side of the patient femur between the patient femur and patient tibia, and a lateral flexion gap (Fl) on the lateral side of the patient femur between the patient femur and patient tibia, with the knee joint in flexion;
selecting a femoral component size for the planned femoral component to be placed on the patient femur, wherein the selecting the femoral component size includes:
using a current size of the planned femoral component, setting an angular position of the planned femoral component based on at least one of flexing or extending the planned femoral component to provide a desired relation as between Fm and Em; and
determining whether to resize the planned femoral component based on determining whether setting the angular position exceeds a flexion threshold or exceeds an extension threshold;
determining whether Em and El remain in the desired extension gap relation;
performing processing based on the determining whether EM and El remain in the desired extension gap relation, wherein based on determining that Em and El remain in the desired extension gap relation, the performing processing includes providing parameters of the planned distal femur resection to a device, the parameters based on the set cutting angle, distal femur resection amounts, planned femoral component rotation, and angular position, and the selected femoral component size; and
building and providing a graphical user interface displaying the provided parameters.
